(19) United States
(12) Patent Application Publication
(10) Pub. No.: US 2006/0074485 A1
(43) Pub. Date: Apr. 6, 2006

(54) Method and apparatus for percutaneous valve repair

(76) Inventor: Fidel Realyvasquez, Palo Cedro, CA (US)

Correspondence Address: HELLER EHRMAN LLP, 275 MIDDLEFIELD ROAD, MENLO PARK, CA, 94025-3506, US

(21) Appl. No.: 11/131,914

(22) Filed: May 17, 2005

Related U.S. Application Data
(60) Provisional application No. 60/572,133, filed on May 17, 2004.

(51) Int. Cl. A61F 2/24; A61B 17/3205; A61B 17/12

(52) U.S. Cl. 623/2.11; 606/108; 606/170; 606/200; 623/2.38; 606/151

(57) ABSTRACT

Methods and apparatus are provided for valve repair or replacement. In one embodiment, the apparatus is a valve delivery device comprising a first apparatus and a second apparatus. The first apparatus includes a heart valve support having a proximal portion and a distal portion and a heart valve excisor slidably mounted on said first apparatus. The second apparatus includes a fastener assembly having a plurality of penetrating members mounted to extend outward when the assembly assumes an expanded configuration; and a heart valve prosthesis being releasably coupled to said second apparatus. The first apparatus and second apparatus are sized and configured for delivery to the heart through an opening formed in a femoral blood vessel. The heart valve prosthesis support is movable along a longitudinal axis of the device to engage tissue disposed between the anvil and the valve prosthesis. The system may include a tent and/or an embolic screen to capture debris from valve removal.

The application claims the benefit of priority from copending U.S. Provisional Application Ser. No. 60/572,133 (Attorney Docket No. 40450-0006) filed May 17, 2004 and fully incorporated herein by reference for all purposes.

### BACKGROUND OF THE INVENTION

1. Technical Field

The invention relates to apparatus and methods for minimally invasive heart valve replacement and is especially useful in aortic valve repair procedures.

2. Background Art

Essential to normal heart function are four heart valves, which allow blood to pass through the four chambers of the heart in one direction. The valves have either two or three cusps, flaps, or leaflets, which comprise fibrous tissue that attaches to the walls of the heart. The cusps open when the blood flow is flowing correctly and then close to form a tight seal to prevent backflow.

The four chambers are known as the right and left atria (upper chambers) and right and left ventricles (lower chambers). The four valves that control blood flow are known as the tricuspid, mitral, pulmonary, and aortic valves. In a normally functioning heart, the tricuspid valve allows one-way flow of deoxygenated blood from the right upper chamber (right atrium) to the right lower chamber (right ventricle). When the right ventricle contracts, the pulmonary valve allows one-way blood flow from the right ventricle to the pulmonary artery, which carries the deoxygenated blood to the lungs. The mitral valve, also a one-way valve, allows oxygenated blood, which has returned to the left upper chamber (left atrium), to flow to the left lower chamber (left ventricle). When the left ventricle contracts, the oxygenated blood is pumped through the aortic valve to the aorta.

Certain heart abnormalities result from heart valve defects, such as valvular insufficiency. Valve insufficiency is a common cardiac abnormality where the valve leaflets do not completely close. This allows regurgitation (i.e., backward leakage of blood at a heart valve). Such regurgitation requires the heart to work harder as it must pump both the regular volume of blood and the blood that has regurgitated. Obviously, if this insufficiency is not corrected, the added workload can eventually result in heart failure.

Another valve defect or disease, which typically occurs in the aortic valve is stenosis or calcification. This involves calcium buildup in the valve which impedes proper valve leaflet movement.

In the case of aortic valve insufficiency or stenosis, treatment typically involves removal of the leaflets and replacement with valve prosthesis. However, known procedures have involved generally complicated approaches that can result in the patent being on cardiopulmonary bypass for an extended period of time.

Applicants believe that there remains a need for improved valvular repair apparatus and methods that use minimally invasive techniques and/or reduce time in surgery. Although known technology have described methods to replace a human aortic valve with a prosthesis, these methods are, however, designed to be used while the patient is on cardiopulmonary bypass and an open aorta technique. It is understood that there are potentially adverse effects from cardiopulmonary bypass. Recently, methods have been introduced to insert a stented aortic valve using percutaneous techniques but, unfortunately, the native aortic valve is left in situ and presently limited to very ill patients not suitable for valve replacement by conventional means. The need remains for further improved methods of valve repair and/or replacement.

### SUMMARY OF THE INVENTION

The present invention provides solutions for at least some of the drawbacks discussed above. Specifically, some embodiments of the present invention provide improved methods for treating various aortic valve ailments. In one embodiment, the present invention provides an alternative technique where the native aortic valve is replaced using a percutaneous technique while the patient is under general anesthesia but without cardiopulmonary bypass assistance. Advantageously, the patient may have a more rapid recovery and improved outcomes using such a percutaneous cardiac surgery technique. In one embodiment, the present technique is intended to be used in patients who are not candidates for conventional aortic valve replacement techniques and would be suited for patients who need aortic valve replacement because of a severely regurgitant aortic valve with thin or fibrotic leaflets and minimal calcification. At least some of these and other objectives described herein will be met by embodiments of the present invention.

In one embodiment, the present invention provides a device for percutaneous delivery of a valve prosthesis for valve repair. The device comprises a valve delivery device having a first apparatus and a second apparatus. The first apparatus includes a heart valve support having a proximal portion and a distal portion and a heart valve excisor slidably mounted on the first apparatus. The second apparatus includes a fastener assembly having a plurality of penetrating members mounted to extend outward when the assembly assumes an expanded configuration; and a heart valve prosthesis being releasably coupled to the second apparatus. The first apparatus and second apparatus are sized and configured for delivery to the heart through an opening formed in a femoral blood vessel. The heart valve prosthesis support is movable along a longitudinal axis of the device to engage tissue disposed between the anvil and the valve prosthesis.

In another aspect of the present invention, a method of valve replacement is provided. The method comprises providing a first apparatus having a valve anvil for supporting valve tissue and a valve leaflet cutter. The method also includes providing a second apparatus having a valve prosthesis, a prosthesis protective cone, and valve fastener assembly. A femoral blood vessel is accessed and a guidewire is inserted to guide the first and second apparatus to a target site in the heart. The first apparatus is advanced along the guidewire in a collapsed configuration where the valve leaflet support is advanced through a valve, wherein the support is positioned below a valve annulus. The first apparatus is expanded at the target site into an expanded configuration so that the valve anvil will engage the valve. The method further includes advancing the second apparatus along the guidewire in a collapsed configuration to the target site until the second apparatus engages an alignment marker indicating that the desired position has been reached; advancing a plunger to expand the valve prosthesis and fastener assembly to engage tissue at the target site, wherein expanding the fastener assembly advances penetrating members on the fastener into valve tissue, the penetrating members being secured in the valve tissue and the valve prosthesis, and act as anchors to hold the valve prosthesis in position; moving the valve leaflet support and valve excisor together to remove leaflets of the valve; and capturing cut valve leaflets in the prosthesis protective cone.

In one embodiment of the present invention, a valve replacement assembly is provided that comprises of a valve cutter and a debris tent positioned over the valve cutter to capture debris created by the valve cutter during tissue removal. The device may include an embolic screen positioned downstream from the debris tent. A valve cutter, debris tent, and embolic screen may all positioned over a catheter. An expandable anvil may be positioned upstream from the cutter to engage a target tissue. The expandable anvil may comprise a plurality of fingers hinged to a central hub. A valve prosthesis may be mounted on a catheter coupled to the valve cutter and the debris tent. A kit may be use that includes a valve replacement or delivery assembly as described.

In another embodiment of the present invention, a method is provided that comprises of accessing a femoral blood vessel and inserting a guidewire to guide valve delivery assembly to a target site in the heart. An anvil portion of the valve delivery assembly may be advanced along the guidewire in a collapsed configuration where the anvil is advanced through a valve, wherein the anvil is positioned below a valve annulus. The method may include expanding anvil at the target site into an expanded configuration so that the anvil will engage the valve. A valve fastener portion of the valve delivery assembly may be advanced along the guidewire in a collapsed configuration to the target site until the valve fastener portion engages an alignment marker indicating that the desired position has been reached, wherein the valve fastener portion includes a valve excisor. The method may include expanding the valve prosthesis and valve fastener portion to engage tissue at the target site, wherein expanding the fastener assembly advances penetrating members on said fastener into valve tissue, said penetrating members being secured in said valve tissue and the valve prosthesis, and act as anchors to hold said valve prosthesis in position. The method may include moving the valve leaflet support and a valve excisor together to remove leaflets of the valve and capturing cut valve leaflets in a prosthesis protective cone. The method may further comprise of positioning an embolic screen downstream from the valve fastener portion to capture debris. The method may include positioning an embolic screen downstream from the valve fastener portion and upstream from arteries of the aortic arch.

A further understanding of the nature and advantages of the invention will become apparent by reference to the remaining portions of the specification and drawings.

### DESCRIPTION OF THE SPECIFIC EMBODIMENTS

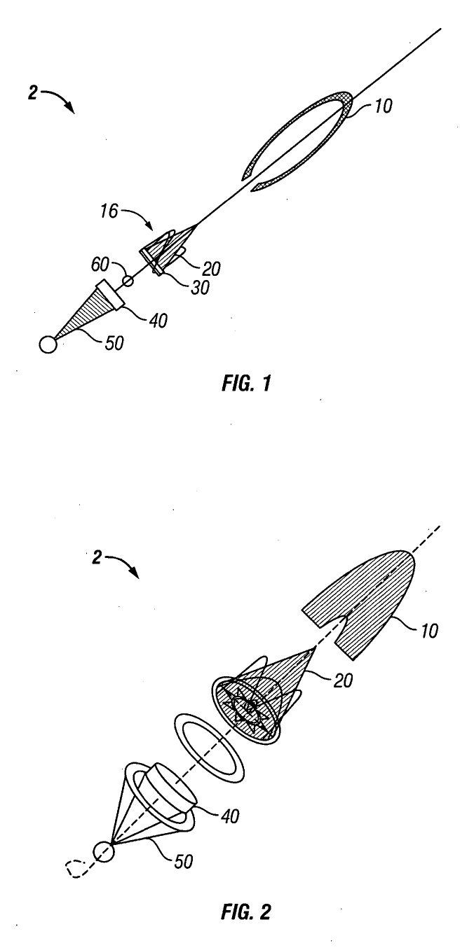

FIG. 1 shows a schematic of one embodiment of the present invention.

FIG. 2 shows an deployed configuration of the embodiment of FIG. 1.

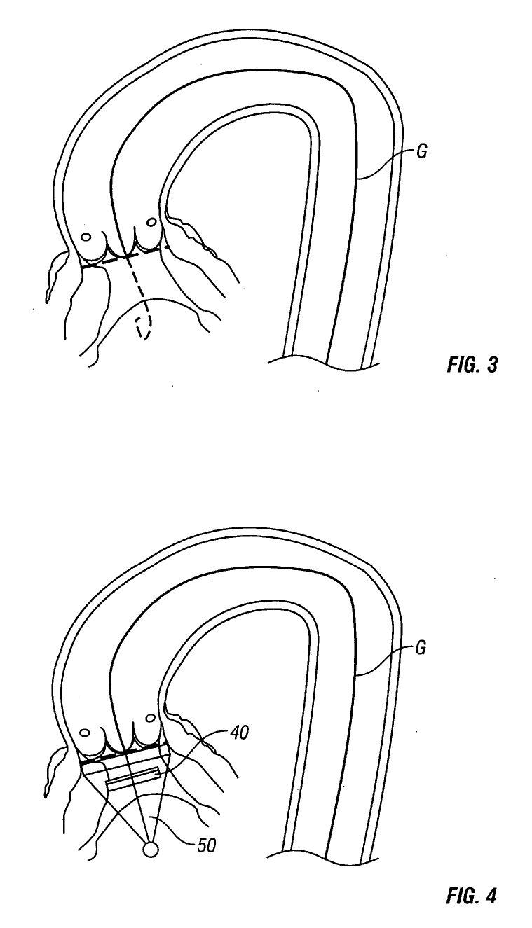

FIGS. 3 through 6 show one method of delivering various components of the present invention into the patient.

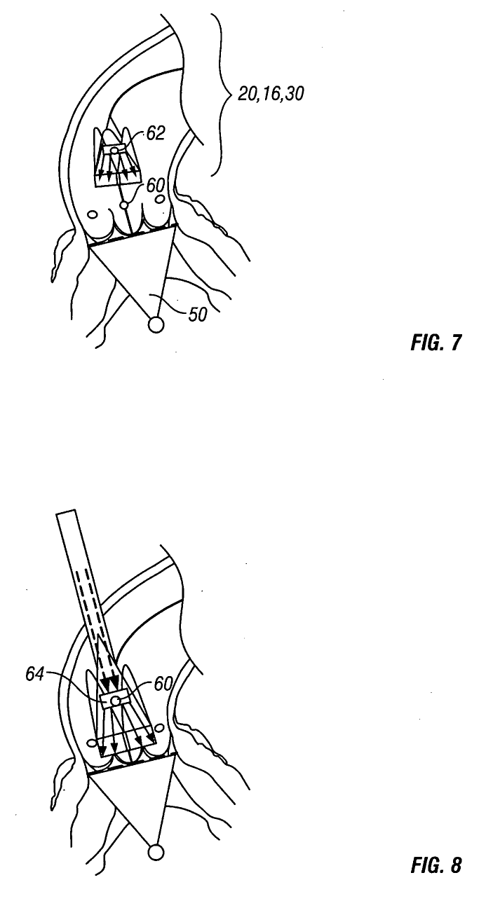

FIGS. 7 through 12 one method of delivering a valve prosthesis and the removal of tissue debris.

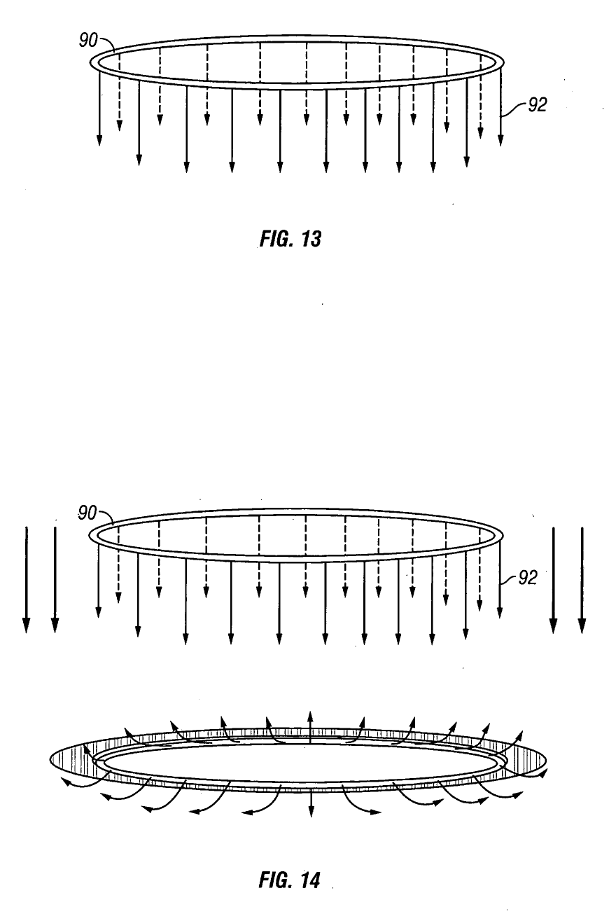

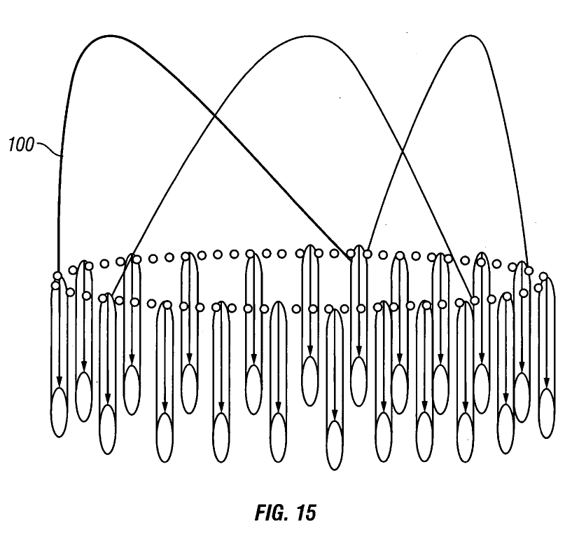

FIGS. 13 and 14 are schematics of a ring with fasteners according to the present invention.

FIGS. 15 through 18 show another embodiment of a ring with fasteners according to the present invention.

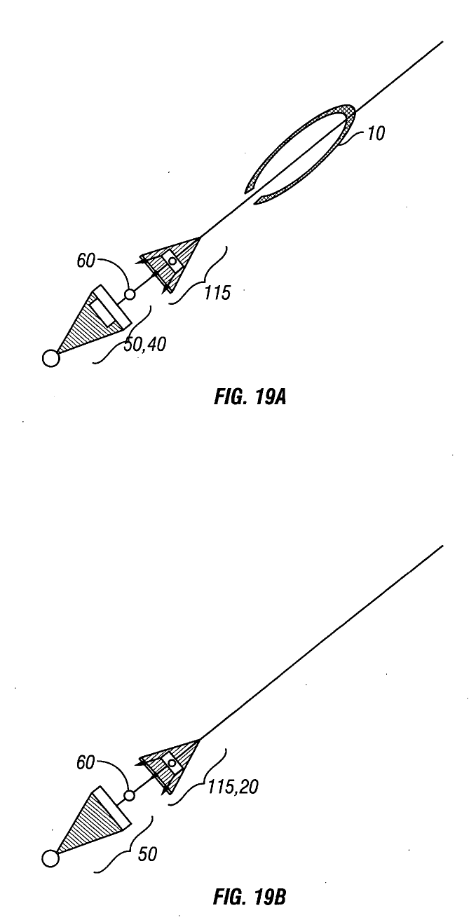

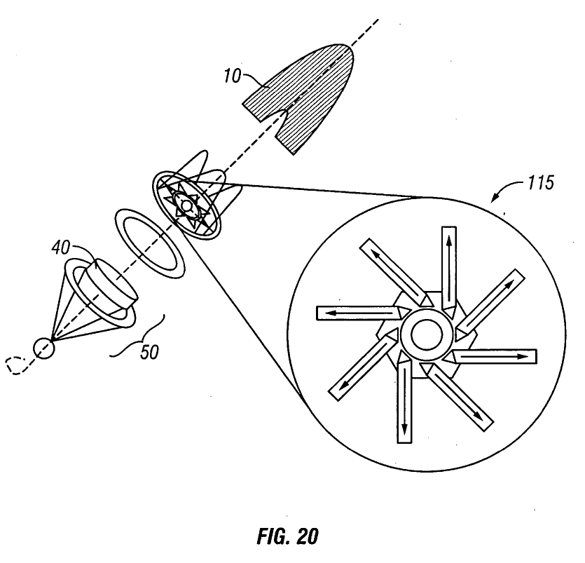

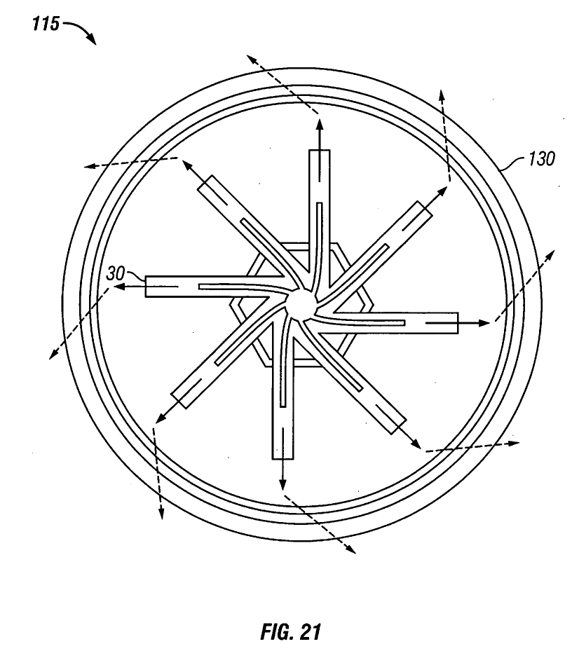

FIGS. 19 and 20 show an embodiment of a valve delivery assembly for use with one embodiment of a radial fastener delivery device.

FIGS. 21 through 23 are various views of a radial fastener delivery device.

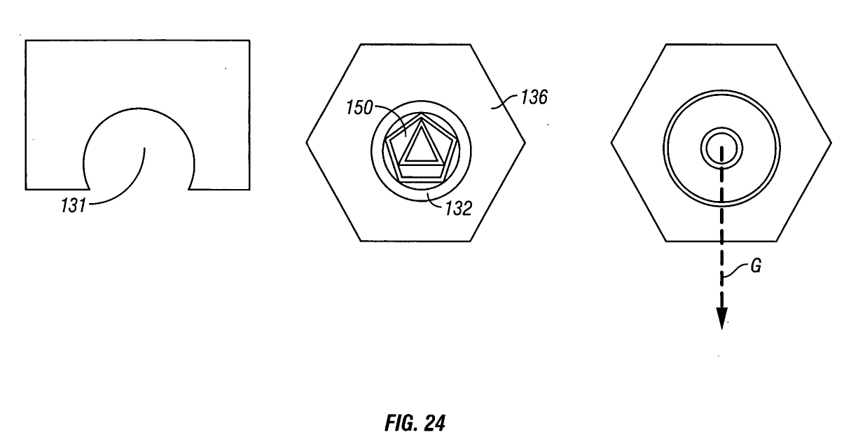

FIGS. 24 through 26 show views of fasteners for use with a radial fastener delivery device.

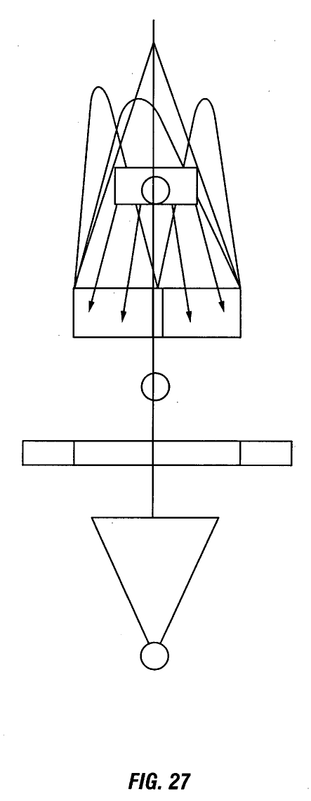

FIG. 27 through 32 show the delivery of valve prosthesis using a radial fastener delivery device.

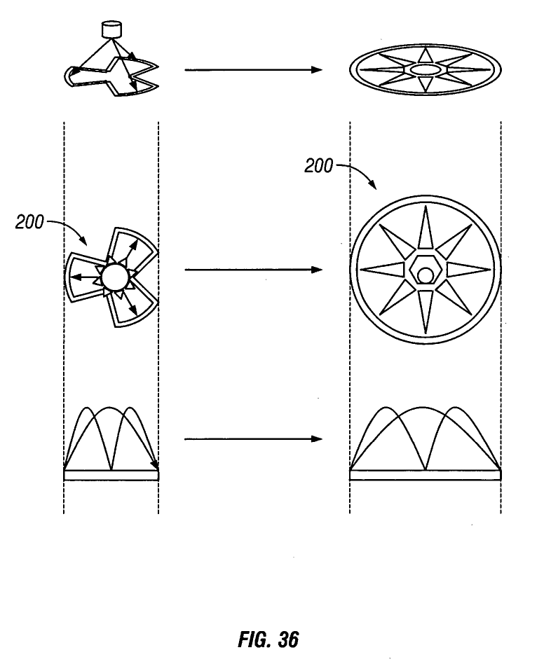

FIGS. 33 and 35 show embodiments of an improved stented valve.

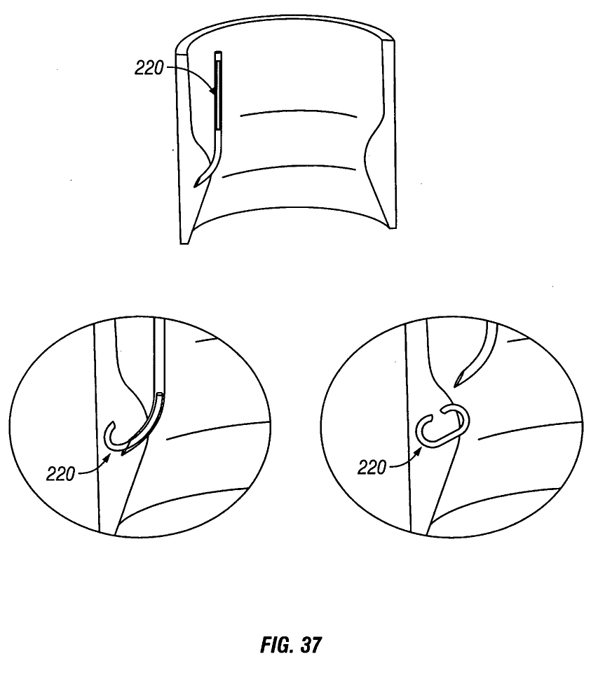

FIG. 36 shows one embodiment of a valve with a collapsible annulus.

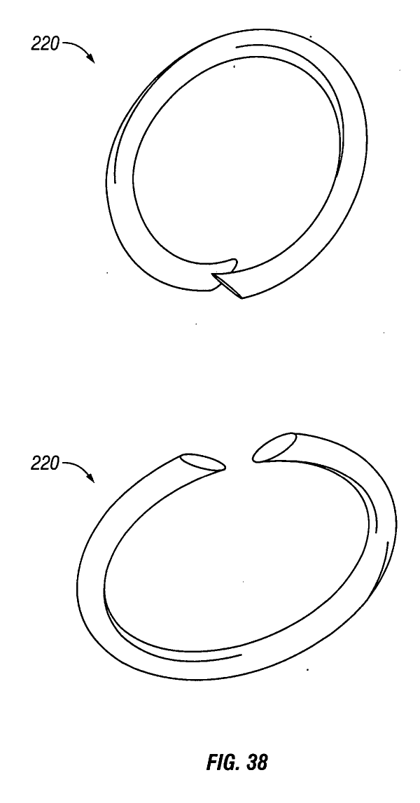

FIGS. 37 and 38 show embodiments of a shape memory fastener.

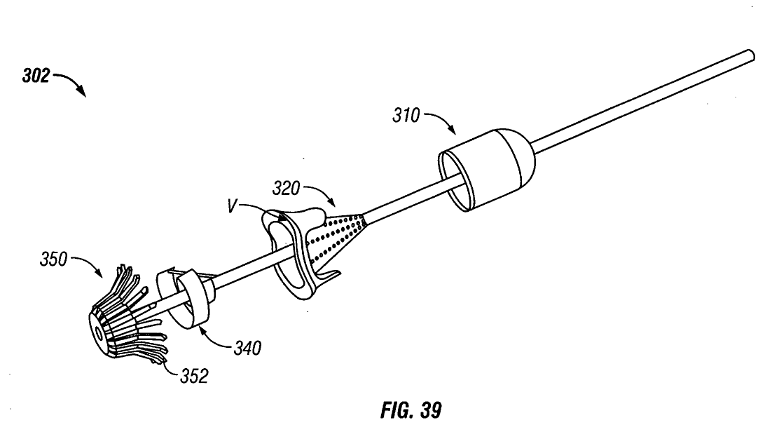

FIG. 39 shows another embodiment of a valve delivery assembly according to the present invention.

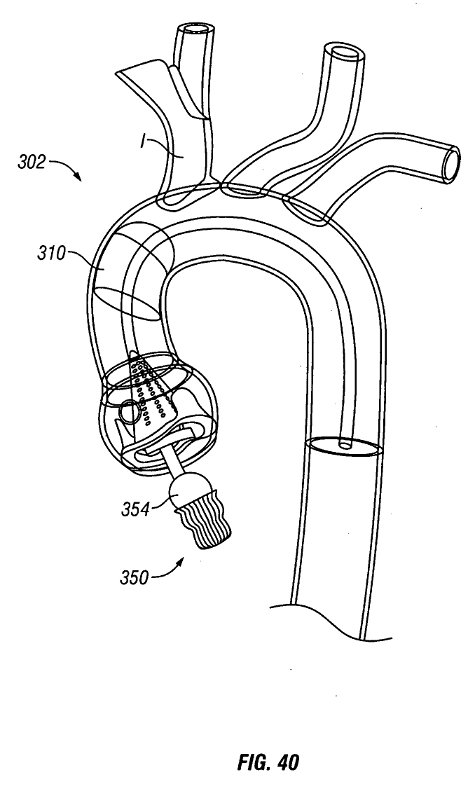

FIGS. 40 through 42 show embodiments of the a valve delivery assembly in the aorta.

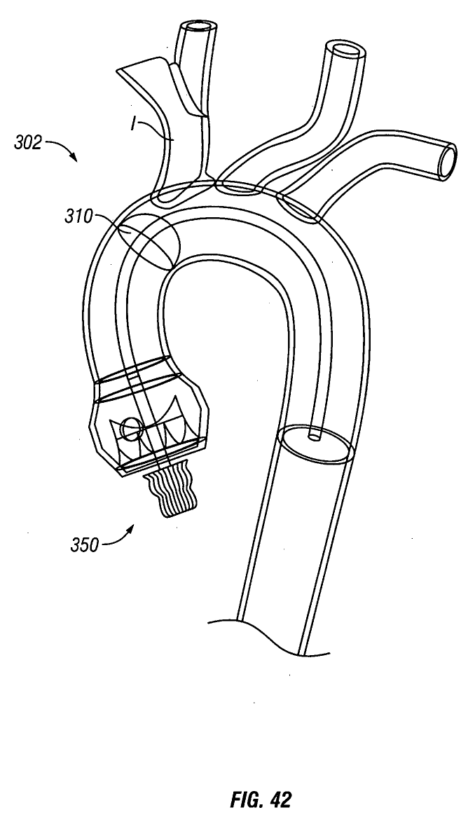

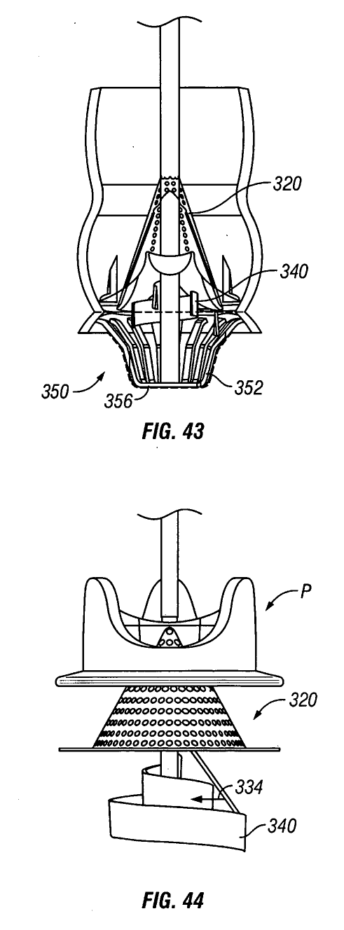

FIGS. 43 through 45 show various views of one portion of the valve delivery assembly.

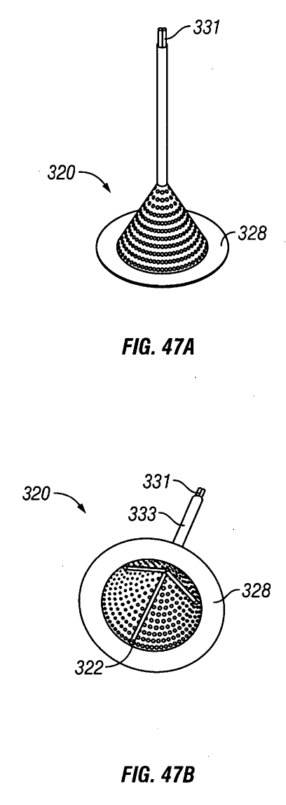

FIGS. 46 and 47 show embodiments of a tent according to the present invention.

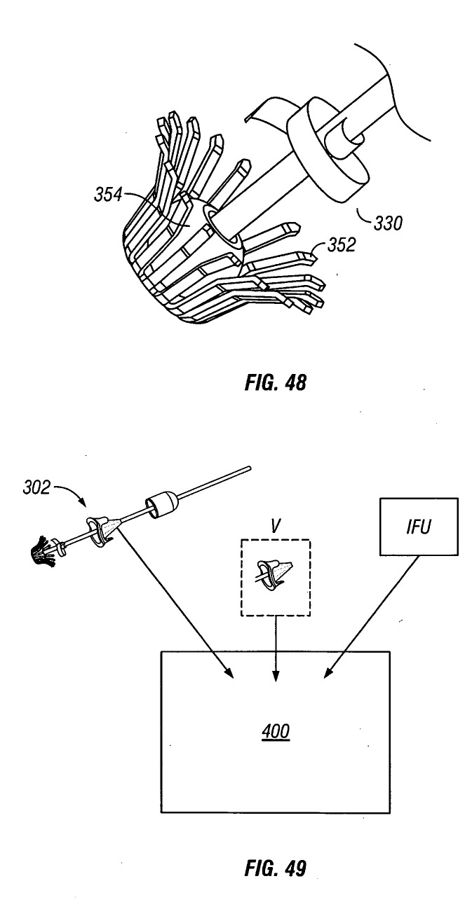

FIG. 48 shows a close up view of one embodiment of a cutter and an anvil according to the present invention.

FIG. 49 show a kit according to the present invention.

### DESCRIPTION OF THE SPECIFIC EMBODIMENTS

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for using an inflatable valve support, this means that the inflatable feature may or may not be present, and, thus, the description includes structures wherein a device possesses the inflatable feature and structures wherein the inflatable feature is not present.

In a prior provisional patent application, there is a description of methods to implant a prosthetic aortic valve using off pump techniques. Embodiments of the present invention now describe methods and improvements to deliver a prosthetic device using a percutaneous approach.

The following is a decryption of the methods and the improvement will be noted when appropriate.

Description: Percutaneous Aortic Valve Delivery System Components

In one embodiment of the present invention, the percutaneous aortic delivery assembly may comprise of five major components as shown in FIG. 1. By way of example and not limitation, the components may include:

1. Embolic aortic screen 10

2. Aortic Valve Outflow Protector (Tent) 20

3. Aortic valve and Fastener Assembly 30

4. Aortic Valve Cutter 40

5. Aortic cone anvil 50

The present embodiment of the invention may also include a fastener assembly and aortic cone anvil engaging device 60. This device 60 may be used to facilitate alignment and positioning of the fastener assembly during deployment of the assembly at the valve. It maybe a physical bump or protrusion. It may also be a radioopaque marker.

FIG. 1 shows the assembly 2 in it pre-deployment state and FIG. 2 shows an exploded view of the various elements of assembly 2 in a deployed configuration. It should be understood that for ease of illustration, these elements are not shown with catheters or other attachments that may be used to guide the elements into position.

As seen in FIGS. 1 and 2, the embolic screen 10 is positioned downstream from the valve replacement elements of assembly 2. The screen 10 will serve to catch any debris that may result from the valve replacement procedure. It may be particularly useful in capturing debris not captured by the tent or generated outside of the cutting area in the tent. By way of example and not limitation, the screen 10 may be made of a variety of materials including Dacron™, Goretex™, bovine pericardium, and/or biologically compatible material for circulatory system. Some other embodiments of the present invention are also shown starting at FIG. 39.

Referring now to FIGS. 3 to 6, one method for using the present embodiment of assembly 2 will now be described. In the present embodiment of the invention, access to the ventricular-arterial junction may be via the common femoral artery. Arterial access will be through percutaneous seldinger techniques. In the present example, an arterial puncture may be performed in the common femoral vein and a guide wire is introduced in a retrograde fashion up the arterial tree and positioned proximal to the left ventricular outflow tract (aortic valve annulus). In some embodiments, a dilator may be used to expand a puncture to sufficient size to introduce.

FIGS. 3 to 6 describes four preliminary stages prior to deployment of the aortic valve prosthesis.

Stage #1 (FIG. 3) shows the guide wire G advanced through the aortic valve and positioned in the left ventricle below the ventriculo-arterial junction.

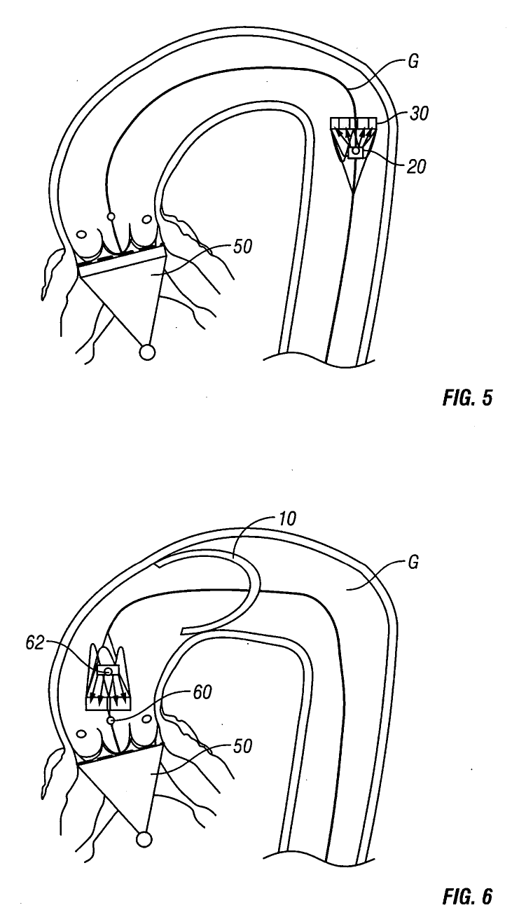

Stage #2 (FIG. 4) shows the aortic cone anvil 50 and cutter 40 in position at, below, above, or near the ventriculo-arterial junction. The cutter 40 is in a retracted position. The cone anvil 50 may be snuggly opposed to the ventricular side of the native aortic annulus.

Stage #3 (FIG. 5) shows the advancement of the aortic prosthesis, the tent 20 and rotational fastener 30 delivery system.

Stage #4 (FIG. 6) shows the embolic screen 10 and aortic valve with its components ready to engage the ball locking mechanism 60. There is mating portion 62 on the aortic valve assembly to facilitate positioning of the valve. The screen 10 is typically positioned upstream of the various arteries in the aortic arch. The flow of blood will help open the screen 10, allowing it to spring open like a parachute when the screen 10 is initially opened. The tent 20 may also be deployed in a similar manner.

It should be understood that these elements described in FIGS. 1-6 may all be integrated on to one catheter. In other embodiments, the elements may be mounted on separate delivery devices and sequentially deployed into the appropriate areas. By way of example and not limitation, they may be mounted on rapid exchange type catheters which may slide over the same guidewire or other catheter.

FIGS. 7 to 12 describe a sequence of steps that result in the engagement of the aortic valve assembly, deployment of a plurality of fasteners, excision of the native aortic valve leaflets and removal of the remaining components.

Referring now to FIG. 7, sequence-1 will now be described. The tent 20, aortic prosthesis 16 and fastener assembly 30 are in their predeployment state. In this embodiment, the rotational fastener delivery system is ready to engage the ball socket mechanism 60. The purpose of the ball socket mechanism is for orientation of the entire assembly to the aortic annulus and the aortic cone anvil.

Referring now to FIG. 8, sequence-2 will now be described. This sequence demonstrates the engagement of the ball socket mechanism 60 created by the downward force provided by a plunger or pusher on the rotational fastener delivery system. This results in the delivery of the aortic prosthesis at the level of the native aortic annulus. As will be discussed in more detail below, the movement of the collar 64 around portion 62 causes the movement of linkages that deploy fasteners outward in a radial fashion, in the same plane as the valve prosthesis. In one embodiment, the downward force on the plunger shaft deploys aortic prosthesis at level of aortic annulus with ball socket mechanism.

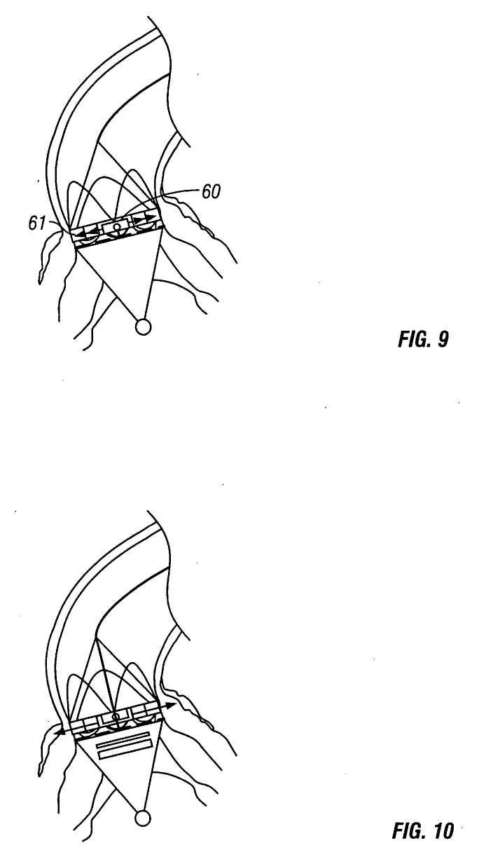

Referring now to FIG. 9, sequence-3 will now be described. The rotational fastener sheaths have been deployed at the level of the annulus and ready for fastener delivery. In some embodiments, the ball socket 60 may be movable or the guidewire G may be movable to as the fastener assembly is expanded. Fastener sheaths have been deployed to the level of the native aortic annulus. The arrows 61 indicate that fasteners may be ready for deployment at level of aortic annulus.

Referring now to FIG. 10, sequence-4 will now be described. The rotational force (indicated by arrow 70) on the hexagonal cable which is attached to the rotational fastener mechanism results in deployment of fasteners into the native aortic annulus and fastening the prosthesis. It should be understood that other cross-sections such as oval, triangular, square, polygonal, keyed, or any single or multiple combination of the above may be used to provide a torque transferring cable. Aortic Cone anvil may collapse to less than internal orifice diameter of aortic prosthesis. It should be understood that the tent and screen may be used with a variety of cutters and valve fasteners. The present description is purely exemplary and is nonlimiting.

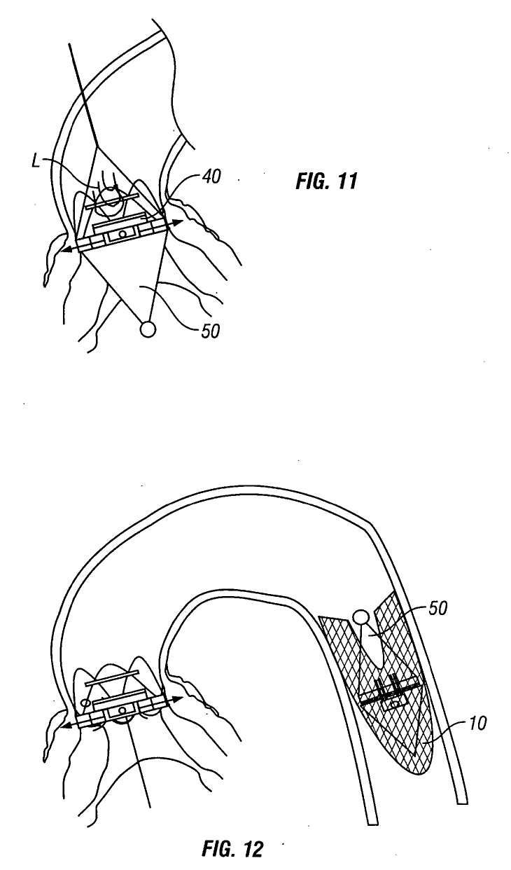

Referring now to FIG. 11, sequence-5 will now be described. Once the fasteners have been deployed, the diameter of the cone anvil is reduced to the size of the cutter. Pulling and rotating the cable which is attached to the cutter 40 results in excision of the native aortic valve leaflets. The valve leaflets L and debris are collected in the cone anvil 50 and the tent 20. The prosthetic leaflets are protected by the tent 20.

Referring now to FIG. 12, sequence-6 will now be described. Once the aortic leaflets L have been excised, the entire assembly is traced within the confines of the embolic screen and extracted from the common femoral artery. The entire embolic screen 10 may, in some embodiments, be sheathed to reduce its outer diameter to facilitate removal from the body. The tent containing valve debris and anvil cone are retracted into the embolic screen and extracted from the common femoral artery. In one embodiment, the screen 10 will close on the tent 20. The anvil 50 may be used to close the open end of the tent 20 and prevent debris from exiting through that end.

Stent Ring Fastener for Prosthetic Aortic Valve Attachment

The present application now describes a Ring Fastener Design. The basic designs consist of a ring and a series of fasteners that are anchored to the ring. The ring is designed so that the valve prosthesis can be anchored to the ring fastener. This device could facilitate attachment of aortic valve prosthesis during an open cardiac surgical procedure and possibly for inserting a valve using a percutaneous approach.

Referring now to the embodiment of FIG. 13, in its pre-deployment configuration, the fasteners are attached to the ring in a perpendicular fashion.

Referring now to FIG. 14, the method of deployment is illustrated. Pressure is applied to the ring fastener 90 and the plurality of fasteners 92 are then directed into the native annulus.

In the present embodiment, two modifications have been added to the ring fastener:

1. Aortic Valve Stent

2. Pre-deployment sheaths.

Referring now to FIG. 15, the figure illustrates the two modifications. The stent 100 modification allow for stented valve prosthesis to be mounted on the ring fastener. Having the proximal fastener attached to the ring simplifies attachment. The distal end becomes the functional portion of the fastener.

The pre-deployment sheaths allow for the nitinol or other material to maintain an inactive phase. The distal end of the sheath has the configuration of a hypodermic needle or lancet with a pointed beveled end to pierce tissue such as but not limited to the annulus.

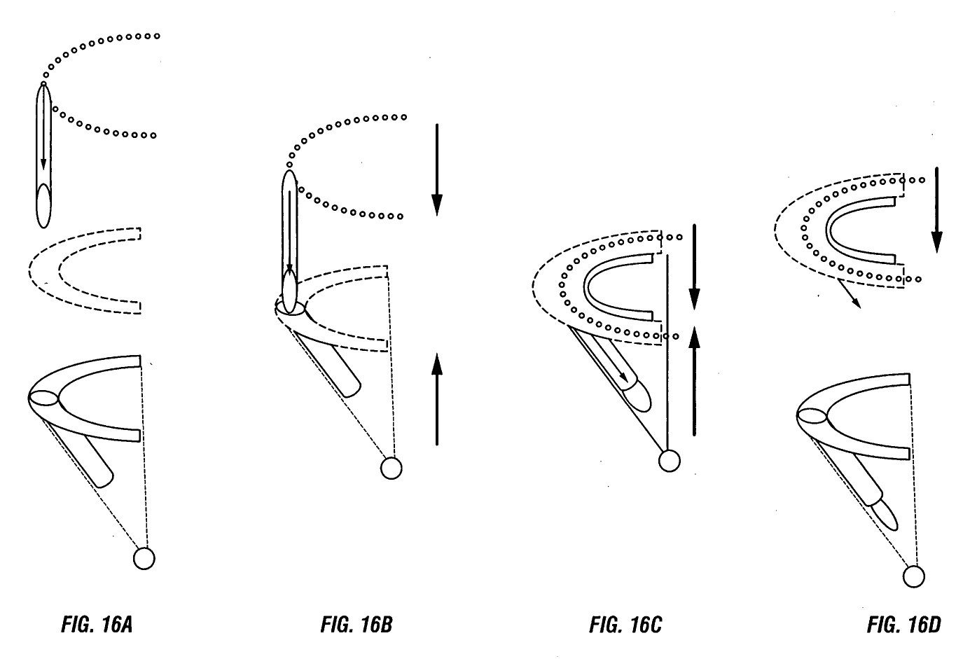

Referring now to FIG. 16, a single fastener has been isolated to more clearly illustrate the mechanism of fastener deployment. In “A”, the fastener I 10, fastener sheath, annulus and cone anvil are in alignment, allowing the fastener to be aligned with the opening of a sheath in the cone anvil. This can be accomplished by providing an orientation mechanism on the shaft of the cone anvil (keying, socket, or other alignment device).

Referring now to illustration “B” in FIG. 16, once the alignment has been accomplished, opposing forces come together to drive the fastener 110 into the native aortic annulus “B”. The fastener sheath 120 is driven into the receiving sheath. Both sheaths then become adhered to one another as shown in illustration “C”. The cone anvil is then pushed away from the aortic annulus releasing the fastener to attach the valve prosthesis to the native aortic annulus “D”.

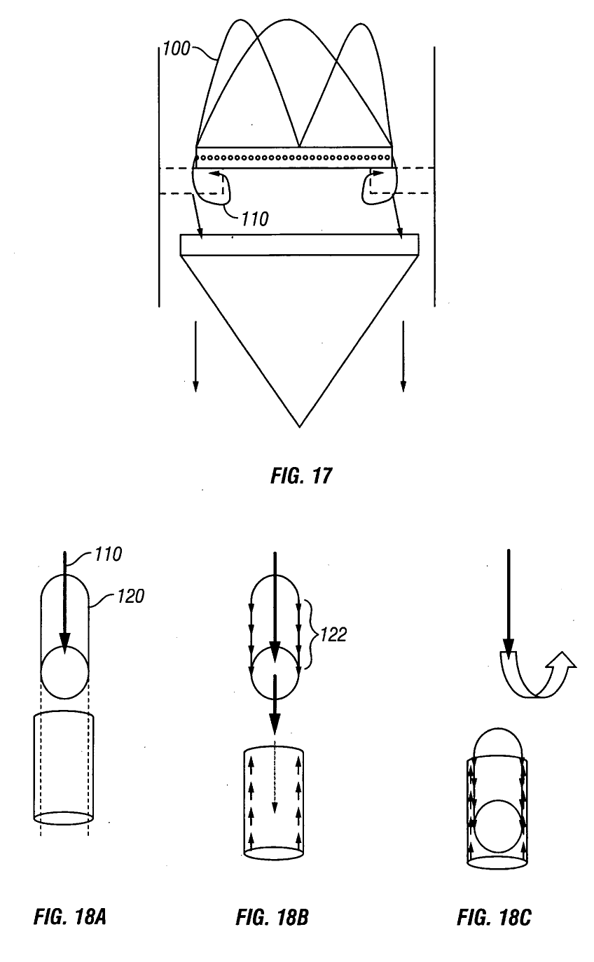

FIG. 17 illustrates the fashion by which the aortic valve prosthesis becomes attached by the fastener 110 after release from the fastener sheath. The shape memory nature of the material used in this embodiment of the fastener allows the fastener to curve and engage the tissue at the valve junction.

Referring now to FIG. 18, this figure illustrates the detail of the fastener deployment sheath 120 and the locking mechanism for retraction of the fastener sheath 120 resulting in fastener deployment. In “B”, a series of arrows in the direction of the fastener, in the fasteners sheath, represent locking tines. The same tines are illustrated on the receiving sheath but are directed in the opposite direction of the fastener. When the fastener sheath slides into the receiving sheath, the tines become interlocked “C”. When the aortic cone anvil 50 is push deeper into the left ventricle, the fastener 110 is retracted from the sheath and the fastener is deployed. It should be understood that other one-way locking mechanisms such as mating wedges or apertures and hooks but be used to secure the fasteners. There may also be one-way stops (wedges or the like) which prevent the sheath from being retracted distally once they have entered the anvil.

Radial Fastener Delivery Device for Percutaneous Aortic Valve Replacement

Previously we describe a percutaneous delivery of aortic valve prosthesis. The fastening system refers is a radial fastener device 115 which deploys a plurality of fasteners 130 from within and in the plane of the prosthetic annulus into the native aortic annulus. FIGS. 19 to 32 show a method similar to that of FIGS. 1-12, but shows in more detail, how the fastener is deployed, along with some other modifications.

Referring now to FIGS. 19A and 19B, one embodiment of present invention will now be described. FIG. 19A shows an embodiment similar to that of FIG. 1. Referring now to FIG. 19B, this image demonstrates the modifications to the percutaneous aortic delivery device to facilitate delivery of an aortic prosthesis using open cardiac surgery techniques. It eliminates the embolic screen 10 and the valve cutter 40.

Referring now to FIG. 20, this image describes the relationship of the radial fastener device 1 15 to the aortic valve prosthesis in a position at the same plane within the prosthetic annulus.

Referring now to FIG. 21, this image describes the relationship of radial fastener device 115 to the prosthetic annulus. Within the articulated sheaths are the pre-deployed fasteners 130. The doted fasteners depict the release of the fasteners from within the sheaths to the prosthetic annuls and the native aortic annulus.

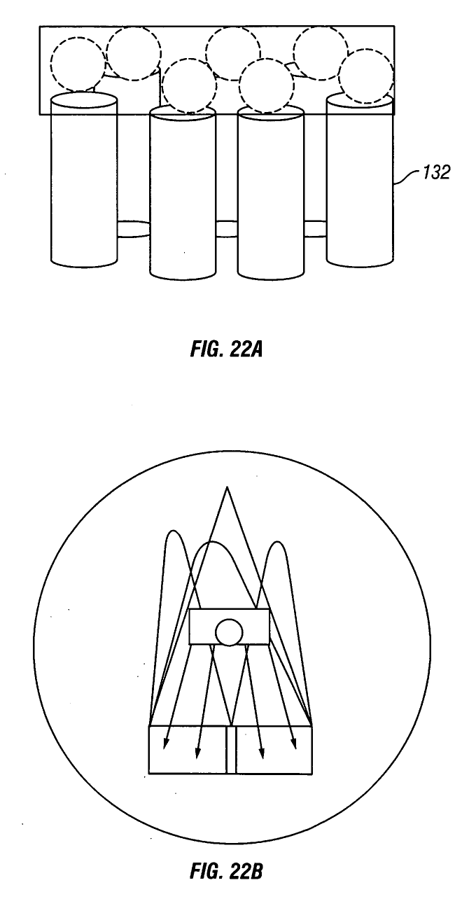

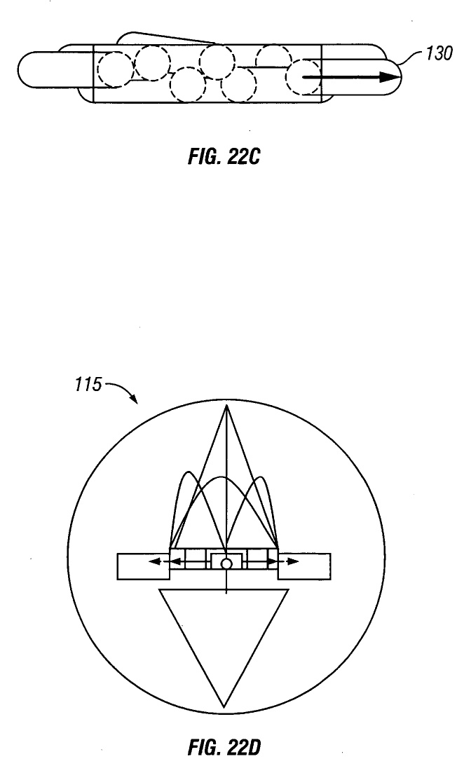

Referring now to FIG. 22, this image depicts the radial fastener system 115 in a collapsed state in illustration “A”. Illustration “B” shows the radial device in relation to the prosthetic annulus and aortic anvil cone. Illustration “C” shows the radial device deployed at the level of the annulus. In this embodiment, the sharpened tips of the fasteners are pointed radially outward and are in the same plane as the ring of the prosthetic device. The deployment of the fasteners is further detailed in illustration “D”.

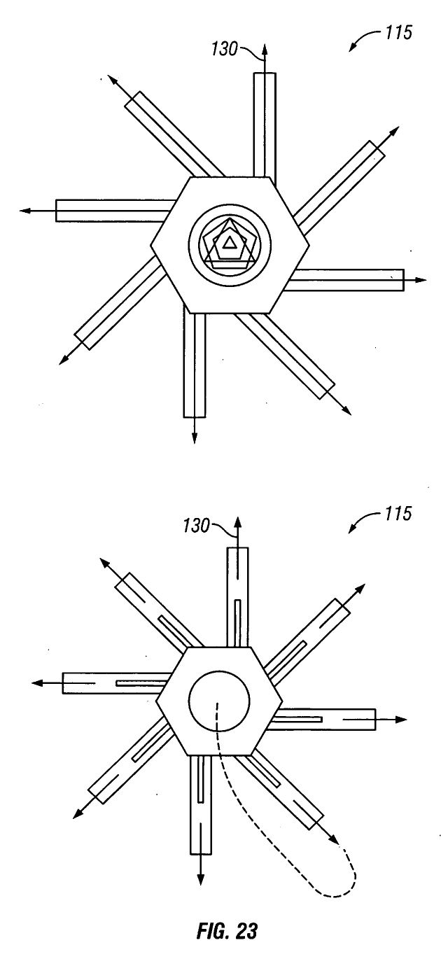

Referring now to FIG. 23, this image shows the superior and inferior surfaces of the radial fastener housing. The “Top” surface reveals the plunger shaft for engagement of the ball socket mechanism 60 and the deployment of the radial arm sheaths of the radial fastener system in a radial fashion prior to fastener deployment. The “Bottom” surface reveals the ball socket which locks and orients the radial fastener above the native aortic annulus prior to deployment of the radial sheaths to the level of the native annulus.

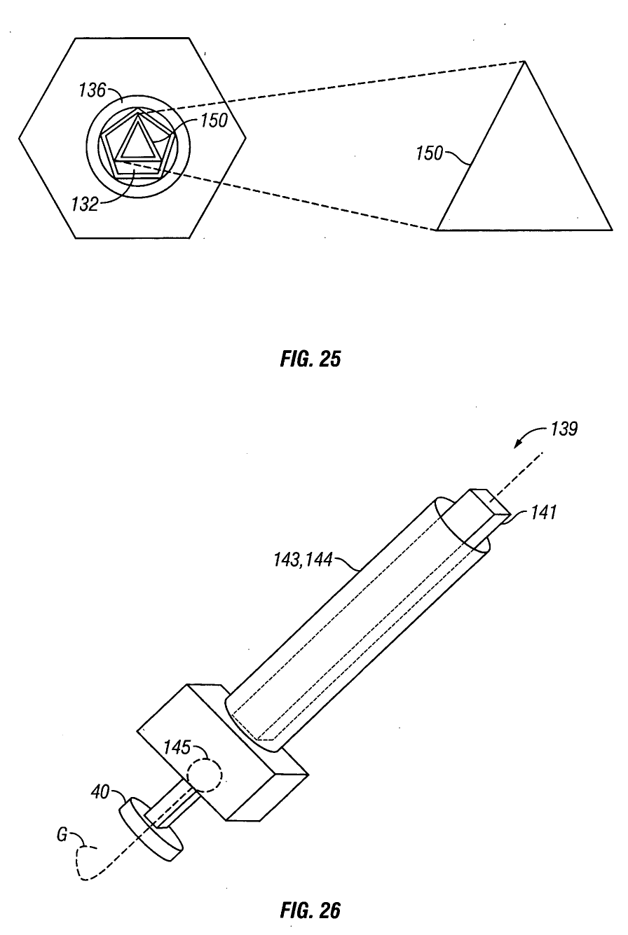

Referring now to FIG. 24, this figure depicts multiple views of the radial fastener housing. Of importance is the introduction of the triangular orientation and alignment device or marker 150 which is better illustrated in FIG. 25. FIG. 24 also shows a plunger 136 for attachment of ball and socket mechanism 131. Fastener deployment cable 132 and guide wire G are also shown.

Referring now to FIG. 25, this image shows the details of the orientation marker 150. This is depicted as a triangle within the pentagon shaped fastener deployment cable. The marker 150 orients the prosthetic valve with the radial fastener device. The apex of the triangle 150 orients the prosthesis and fastener system to the anterior commissure of the native aortic annulus. This will assist in correct alignment and orientation of the prosthetic valve and fastener device at the level of the native aortic annulus. The marker may be colored, such as red, blue, or other color as desired.

Referring now to FIG. 26, this image demonstrates the shaft design for the radial fastener deployment mechanism 139. The square or pentagonal shaft lies within the plunger shaft 143 for radial sheath 144 deployment of the fastener system. The plunger shaft 143 is responsible for engaging the ball/socket mechanism 145 and for deployment of the radial sheaths 144 to the level of the native aortic annulus.

In addition, it is also attached to the cutter 40. Following deployment of the prosthetic valve, proximal traction and rotation on the hexagonal shaft results in excision of the native aortic valve leaflets. Rotation of the hexagonal shaft or cable 141 during deployment of the radial fasteners will not cause premature excision of the leaflet since the cutter in within the confines of the cone anvil during this sequence. FIG. 26 also shows a radial fastener housing 140 and socket 145.

The following is a sequence of steps that introduce, fasten and seat the prosthetic aortic valve.

Referring now to Sequence-1 in FIG. 27, the tent, aortic prosthesis and fasteners are in their predeployment state. This radial fastener delivery system is ready to engage the fall socket mechanism. The purpose of the ball socket mechanism is for orientation of the entire assembly to the aortic annulus and the aortic cone anvil.

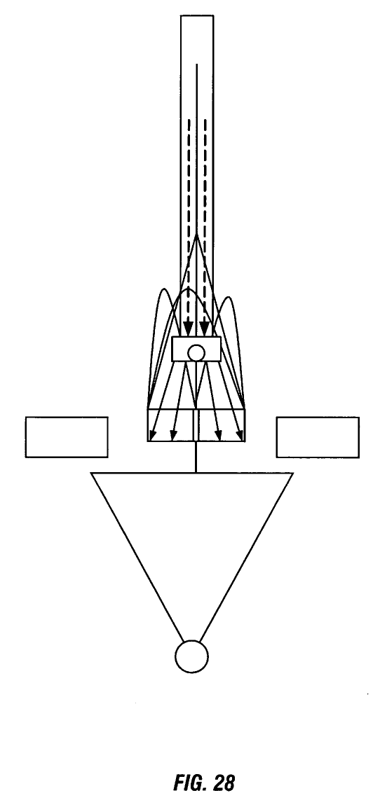

Referring now to Sequence-2 in FIG. 28, this sequence demonstrates the engagement of the ball socket mechanism 131 created by the downward force on the radial fastener delivery system by the plunger shaft 143. This results in the delivery of the aortic prosthesis at the level of the native aortic annulus.

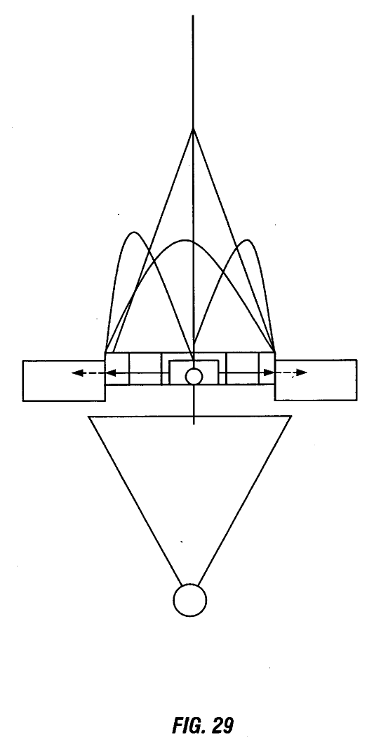

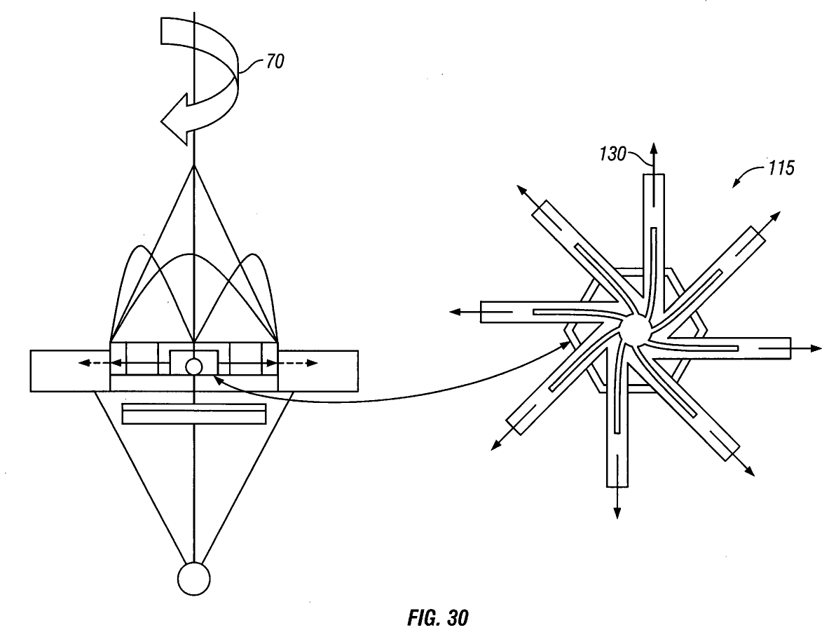

Referring now to Sequence-3 in FIG. 29, the radial fastener sheaths have been deployed at the level of the annulus and ready for fastener delivery.

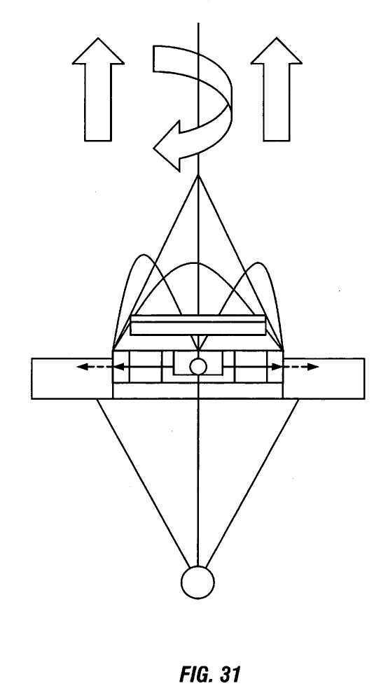

Referring now to Sequence-4 in FIG. 30, the rotational force (arrow 70) on the hexagonal cable which is attached to the rotational fastener mechanism results in deployment of fasteners into the native aortic annulus and fastening the prosthesis.

Referring now to Sequence-5 in FIG. 31, once the fasteners have been deployed, the diameter of the cone anvil is reduced to the size of the cutter. Proximal traction and rotation on the cutter pentagon cable results in excision of the native aortic valve leaflets. The valve leaflets and debris are collected within the confines of the cone anvil and the tent. The prosthetic leaflets are protected by the tent as the cone anvil is extracted.

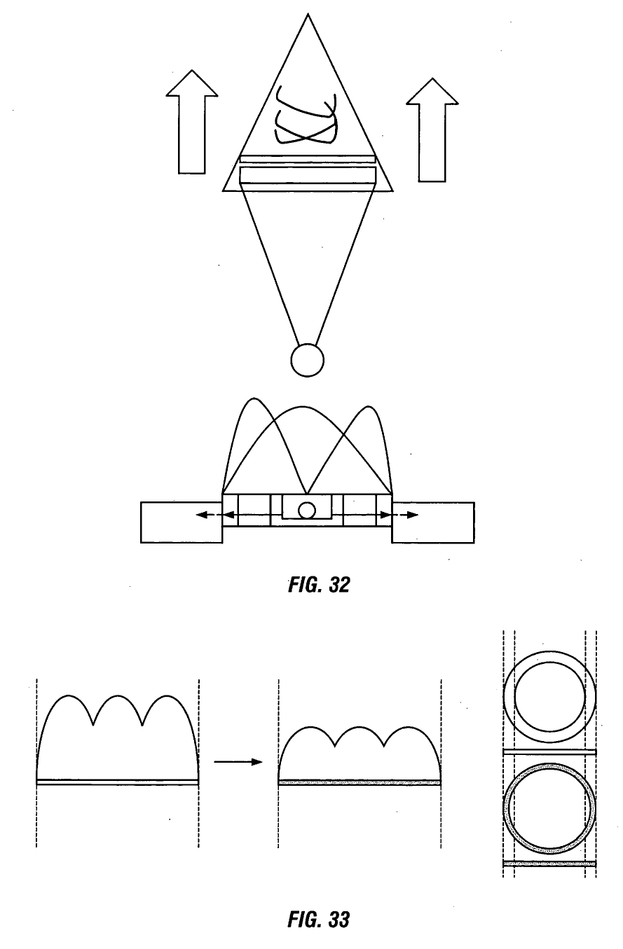

Referring now to Sequence-6 in FIG. 32, once the aortic is seated and attached, the tent, cutter and cone anvil are extracted from the outflow tract through the orifice of the prosthetic aortic valve. The excised native aortic valve leaflets and any other debris are trapped within the tent and aortic cone anvil.

Sewing Ring Modification for the Prosthetic Aortic Valve

Current methods for implantation of heart valve prosthesis involve using well established synthetic suturing techniques. Of major importance to accomplish the fastening of the prosthesis to the native valve annulus is the sewing ring on the prosthesis. The introduction of fastening technology will require modifications of conventional heart valve prosthesis to simplify fastening of prosthetic heart valve to the native annulus. The appropriate modifications include the elimination of the sewing ring and redesigning of the Stent mechanism for heart valve prosthesis. We previously describe the “Ring fastener” and the modification of adding a stent 100 to the ring. This would accomplish two things:

1. The stent 100 with attached fasteners could simplify attachment of aortic prosthetic valve during open cardiac surgical or percutaneous procedures.

2. The stented ring fastener could be made of a material such as but not limited to nitinol with collapsible characteristics that would allow the introduction of the heart valve prosthesis and its anchoring mechanism to be introduced through a catheter.

Referring now to FIG. 33, this image illustrates the advantages of eliminating the sewing ring 180 from the prosthetic annulus. The immediate result would be the implantation of a larger prosthetic valve size for a given native annulus size. This could have significant hemodynamic benefits to patients by increasing effective valve orifice area.

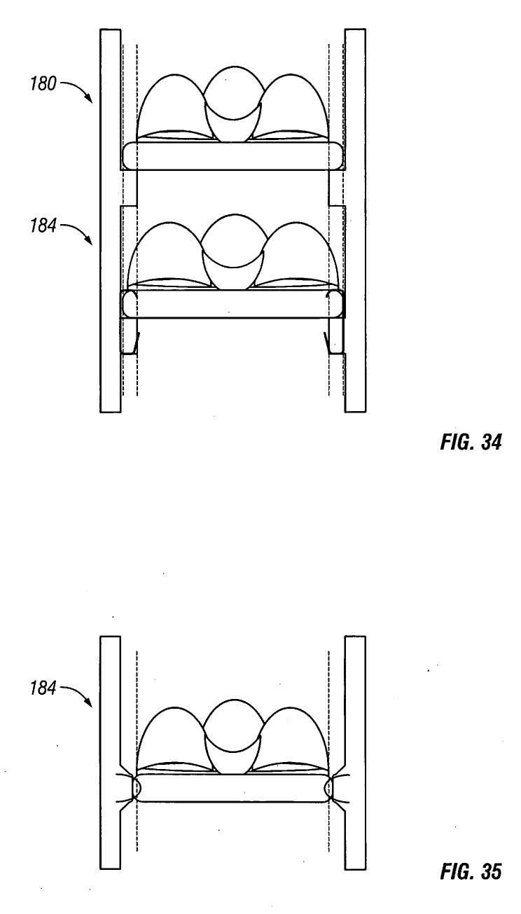

Referring now to FIG. 34, the image illustrates Surpra-Annular Seating of Heart Valve Prosthesis. This image shows the stented prosthesis with a sewing ring “A” and Stented prosthesis 184 without a sewing ring “B”. The valve circumference “B” is greater than “A”. This would translate into a larger effective orifice area in the valve prosthesis without a sewing ring.

Referring now to FIG. 35, the image illustrates Intra-Annular Seating of Heart Valve Prosthesis. A similar result as illustrated in FIG. 34 would occur if the heart valve was implanted in an intra-annular location.

Referring now to FIG. 36, the modification provided by eliminating the sewing ring will facilitate fastener designs.

In “A” the prosthetic annulus is collapsed as illustrated by the red bent segments 200. Attached is the radial fastener deployment device. When the fastener sheaths are deployed, the prosthetic annulus expands when the segments 200 assume a non-folded configuration and expand to form the full circumference of the ring, as seen in illustration B. Illustration C shows a side view of the difference in diameter between a folded and unfolded/expanded configuration.

Referring now to FIGS. 37 and 38, this is the design for one embodiment of the clip fastener 220 for use with the present invention. The concept is basically a needle that pierces the tissue (native aortic annulus). Once you deploy the distal portion of the clip into the annulus, the user pulls back on the needle to deploy the proximal portion of the clip which would then attach to the prosthetic annulus. The present invention shows one embodiment which forms a C shaped device when fully released from its pre-deployment sheath.

Referring now to FIG. 39, a still further embodiment of the present invention will now be described. FIG. 39 shows a fully assembled valve assembly 302 for use in percutanouesly delivering a valve to a target site. The present embodiment may include an embolic screen 310, a tent 320, a valve prosthesis V, a cutter 340, and an anvil 350. For ease of illustration, the fastener assembly 330 is not shown in detail as it should be understood that the tent 320 and the anvil 350 may be used with a wide variety of valve fastening devices and is not limited by any particular design. It should be understood that these elements described in FIG. 39 may all be integrated on to one catheter. In other embodiments, the elements may be mounted on separate delivery devices and sequentially deployed into the appropriate areas. By way of example and not limitation, they may be mounted on rapid exchange type catheters which may slide over the same guidewire or other catheter.

The anvil 350 is made to expand and engage the tissue at the target site. In the present embodiment, the anvil 350 may have a plurality of fingers 352 that act as support elements. These fingers 352 are coupled to a central disc 1234. FIG. 39 shows the anvil 350 in an expanded configuration. A shaped plunger member 354 is inserted into the center of the plurality of fingers 352 and the shaped plunger member 354 has a circumference sufficient to deflect the fingers 352 to a position where the fingers are pushed radially outward. By way of example and not limitation, the shaped plunger member 354 may be rounded as shown in FIG. 39 (and more clearly in FIG. 40) or it may be, but is not limited to, shapes such as spheres, cones, wedges, cubes, polygons, or any single or multiple combination of the above. As seen in this embodiment, the anvil 350 is expanded by drawing the fingers 352 around the ball or pushing the ball into the anvil 350. Although not limited to the following, the fingers 352 may be made from nickel titanium alloy, stainless steel or polymer. In other embodiments, the anvil 350 may have a hinge configuration with parts that may be articulated to expand.

Hinged fingers 352 when in their undeployed position will remain at its minimum radial position to allow passage through the prosthetic valve opening once the tissue engagement device is passed through the valve or the aorta. The articulating hinged fingers can then be deployed to a larger radial configuration to support the tissue. In some embodiments, the expandable device will contact the device to hold it in position. The device may include a support surface to contact the tissue. In some embodiments, the support surface may be used to align or stop the fastener housing.

In some embodiments, the fingers 352 may be coupled together by a mesh material such a DARON, Dacron, a firm rubber substance, GORTEX, any combination of the above, or similar substances to capture debris that may be created by the valve repair procedure. In some embodiments, the fasteners will align to extend outward in the gaps between fingers 352 so that the fingers do not interfere with deployment of the fasteners.

Referring now to FIG. 40, the assembly 302 is shown deployed in the aorta and extending to access the aortic valve. As seen in FIG. 40, the embolic screen 310 may be positioned upstream of the innominate, left common carotid, and left subclavian arteries. This prevents the debris from the valve replacement process from exiting into the circulatory system from these arteries. In some other embodiments the screen 310 may fully or at least partially cover the innominate 1 or other artery. The screen 310 may allow blood to flow through, but prevents debris from flowing through. FIG. 40 also shows that the plunger 354 has not fully engaged the anvil 350 to expand the anvil from its unexpanded configuration to an expanded configuration.

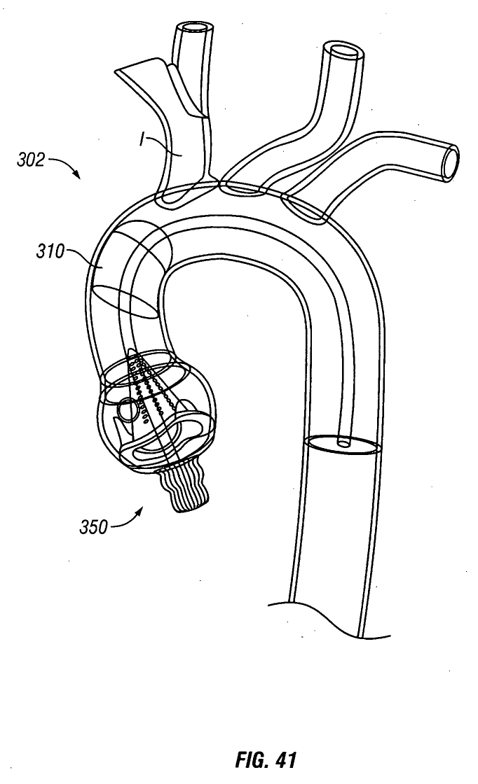

Referring now to FIG. 41, the assembly 302 is shown with the plunger 354 engaged to expand the anvil 350. The tent 320 is also more clearly shown in FIG. 41. The tent 320 serves capture the large elements or portions of debris that may be created during valve leaflet removal. The tent 320 is collapsible and can be used to contain debris for removal. In some embodiments, the tent 320 has larger openings while the screen 310 has finer openings that trap smaller debris.

Referring now to FIG. 42, a simplified version of the assembly 302 is shown.

The screen 310 is shown with a different shape. It is more cup shaped with shorter side walls. It should be understood that the screen 310 may assume a variety of shapes including cone shaped, disc-shaped, or polygonal in shape. The screen 310 may have cross-section that is round, oval or other shaped. The screen 310 may be configured to fully engage circumference of the wall of the blood vessel in a manner or shape that prevents debris from passing around and slipping by the screen 310. The screen 310 may create a seal against the wall of the blood vessel.

Referring now to FIG. 43, more detailed cross-sectional view of the valve leaflet cutting assembly. As seen in FIG. 43, one embodiment may use a spiral cutter 340 to remove the diseased valve leaflets. The tent 320 may be included to contain the debris created by cutter 340. In one embodiment, the tent 320 may be made of substantially the same material as that used for the screen 310. It should be noted that in FIG. 43, a mesh or other material may optionally be spaced between or over fingers 352 as indicated by dotted line 356 to trap debris therein.

Referring now to FIG. 44, shows a close-up view of one embodiment of the tent 320 positioned inside the valve prosthesis P. Cutter 340 is also shown. Arm 332 on the cutter 340 is movable to expand the cutter by controlling the outer circumference of cutter 340. When the arm is brought towards the centerline as indicated by arrow 334, the cutter will spiral upon itself into an unexpanded configuration.

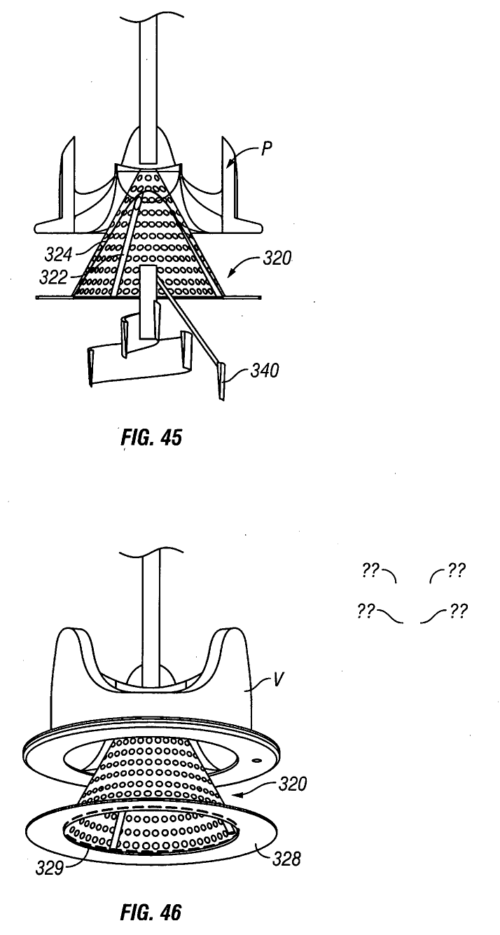

Referring now to FIG. 45, a cross-sectional view of the elements of FIG. 44 is shown. FIG. 45 shows that the current embodiment of the tent 320 may include supports 322 inside the tent 320. The supports 322 are movable as indicated by arrow 324 to expand or collapse the tent 320. In one embodiment, the supports 322 are hinged and are moved from expanded and unexpanded positions through wires (not shown) which are movable and extend along a catheter outside the body of the patient. In other embodiments, the supports 322 may have shape memory or are spring actuated to extend to an expanded configuration when a sheath (not shown) is removed from covering the supports 322.

Referring now to FIG. 46, yet another view is provided of the tent 320 and the valve prosthesis. As seen in this figure, the tent 320 includes an annulus 328. This annulus 328 will be secured again the valve tissue or other target tissue during cutting.

This will help keep the tent 320 expanded and open to catch debris. In the present embodiment, the annulus 328 will be secured with the valve prosthesis V to the target tissue. The tent 320 will tear away along a tear line 329. After the valve leaflets are cut and the tent 320 is ready for removal, the tent 320 may be detached from the annulus 328 by pulling or retracting back on the tent 320 to cause separation at the tear line 329. The anvil 350 will be used to cover any debris and prevent them from escaping out the open end of the tent 320.

FIGS. 47A and 47B also show additional views of one embodiment of the tent 320. FIG. 47A shows that supports 322 are coupled to rod portions 331 that extend proximally along a shaft portion of the device. The rod portions 331 may be movable to extend or retract the supports 322 from expanded and unexpanded positions. FIG. 47B shows that the supports 322 extend along the inside of the tent 320 and into the shaft 333 of the device.

FIG. 48 shows yet another view of the fingers 352, the plunger 354, and cutter 340. During use, the anvil 50 defined by the fingers 352 and plunger 354 will engage tissue. The user will seat the prosthetic valve over the existing valve with the help of the anvil 50. The user will then use cutter 340 to cut the old valve out. The user will then collapse anvil 350 and the tent to retract them. Some embodiments may use the anvil to compress the tent.

Referring now to FIG. 49, one embodiment of a kit according to the present invention will now be described. A package 400 may be used to contain instructions for use (IFU) describing a method for valve delivery. The package 400 may also include the assembly 302. The valve prosthesis V may be mounted on assembly 302 or it may be a separate device as shown in phantom in FIG. 49.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, a prosthetic valve or a graft may be premounted on to the apparatus. With any of the above embodiments, the apparatus may be configured to be delivered percutaneously or through open surgery. With any of the embodiments herein, the devices may be attached by a variety of techniques including sutures, preattached sutures and needles, shape memory clips that will engage tissue, anchors, other fastener device, or any combination of the above. It should be understood that the present invention may be adapted for use on other valves throughout the body. Embodiments of the present invention may be used with stented, stentless, mechanical, or other valves. Some embodiments may be used in open surgery or for off-pump, minimally invasive techniques. The elements shown in FIGS. 1 and 2 may be fully enclosed in one catheter or separate catheters. These catheters may have sheaths that retract to reveal the active portions of the anvil and the cutter to allow for deployment.

The catheter may be coaxially mounted about the guidewire or in some embodiments, they may have extensions or arms that follow the guidewire while the catheter itself is spaced apart from the guidewire. With any of the embodiments, there may be alterative embodiments with only a tent and no embolic screen and vice versa. With any of the above embodiments, there may be more than one tent or more than one embodiment screen. Some embodiments may have two, three, or four embolic screens. Some may have embolic screens made of more than one piece. With any of the embodiments, it should be understood that the embolic screen and tent may be used with cutters of other configurations and valve fasteners of other configurations than those shown herein.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited. U.S. Provisional Application Ser. No. 60/572,133 (Attorney Docket No. 40450-0006) filed May 17, 2004 is fully incorporated herein by reference for all purposes.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A valve replacement assembly comprising:
a valve cutter; and
a debris tent positioned over the valve cutter to capture debris created by the valve cutter during tissue removal.

2. The device of claim 1 further comprising an embolic screen positioned downstream from the debris tent.

3. The device of claim 2 wherein the valve cutter, debris tent, and embolic screen are all positioned over a catheter.

4. The device of claim 1 further comprising an expandable anvil upstream from the cutter to engage a target tissue.

5. The device of claim 4 wherein the expandable anvil comprises a plurality of fingers hinged to a central hub.

6. The device of claim 1 further comprising a valve prosthesis mounted on a catheter coupled to the valve cutter and the debris tent.

7. A method of valve replacement, the method comprising:
providing a first apparatus having a valve anvil for supporting valve tissue and a valve leaflet cutter;
providing a second apparatus having a valve prosthesis, a prosthesis protective cone, and valve fastener assembly;
accessing a femoral blood vessel and inserting a guidewire to guide the first and second apparatus to a target site in the heart;
advancing the first apparatus along the guidewire in a collapsed configuration where the valve leaflet support is advanced through a valve, wherein the support is positioned below a valve annulus;
expanding said first apparatus at the target site into an expanded configuration so that said valve leaflet support will engage said valve;
advancing the second apparatus along the guidewire in a collapsed configuration to the target site until the second apparatus engages an alignment marker indicating that the desired position has been reached;
advancing a plunger to expand said valve prosthesis and fastener assembly to engage tissue at the target site, wherein expanding the fastener assembly advances penetrating members on said fastener into valve tissue, said penetrating members being secured in said valve tissue and the valve prosthesis, and act as anchors to hold said valve prosthesis in position;
moving said valve leaflet support and valve excisor together to remove leaflets of the valve; and
capturing cut valve leaflets in the prosthesis protective cone.

8. The method of claim 7 wherein the first apparatus and second apparatus are each mounted on separate catheters.

9. The method of claim 7 wherein the first apparatus and second apparatus are each mounted on the same catheter.

10. The method of claim 7 wherein the second apparatus includes an embolic screen that is deployed downstream from the target site to capture debris from the valve.

11. The method of claim 7 wherein the penetrating members of the fastener assembly expand outward, each on a curved outward path.

12. The method of claim 7 wherein the marker comprises a ball socket mechanism that mates with a socket on the fastener assembly.

13. The method of claim 7 wherein prosthesis includes a stent.

14. The method of claim 7 further comprising using a sheath to maintain said second apparatus in the collapsed configuration.

15. The method of claim 7 further comprising rotating said plunger to extend the penetrating members from the fastener assembly.

16. The method of claim 7 further comprising attaching said valve apparatus at the ventriculo-arterial junction.

17. The method of claim 7 further comprising driving said penetrating members through the valve prosthesis to anchor the prosthesis to the target tissue.

18. A valve delivery device comprising:
a first apparatus comprising
a heart valve support having a proximal portion and a distal portion;
a heart valve excisor slidably mounted on said first apparatus;
a second apparatus comprising
a fastener assembly having a plurality of penetrating members mounted to extend outward when the assembly assumes an expanded configuration;
a heart valve prosthesis being releasably coupled to said second apparatus;
said first apparatus and second apparatus being sized and configured for delivery to the heart through an opening formed in a femoral blood vessel; and
said heart valve prosthesis support movable along a longitudinal axis of the device to engage tissue disposed between the anvil and the valve prosthesis.

19. The device of claim 18 wherein the first apparatus is a catheter with an elongate portion, a distal end and a proximal end.

20. The device of claim 18 wherein the second apparatus is a catheter with an elongate portion, a distal end and a proximal end.

21. The device of claim 18 further comprising a guidewire.

22. The device of claim 18 further comprising ball socket mechanism on the second apparatus to indicate when the fastener assembly is properly positioned for expansion.

23. The device of claim 18 further comprising a plunger longitudinally slidable to push a collar on the fastener assembly to expanding the assembly into an expanded configuration and advance penetrating members into surround tissue.

24. The device of claim 18 further comprising a pericardial tent positioned to capture valve leaflets between the tent and the valve excisor.

25. The device of claim 18 further comprising an embolic screen mounted on said second apparatus.

26. The device of claim 18 further comprising a pericardial tent on said second apparatus and formed of a mesh and positioned to capture valve leaflets between the tent and the valve excisor.

27. The device of claim 18 further comprising an embolic screen mounted to be slidably delivered over the second apparatus.

28. The device of claim 18 wherein said penetrating members are made of nitinol.

29. A fastener assembly comprising:
a ring having a plurality of foldable portions and a plurality of non-folding portions; and
a plurality of penetrating members ejectably mounted on the ring to extend radially outward.

30. The device of claim 29 wherein said penetrating members are made of nitinol.

31. The device of claim 29 further comprising a plurality of sheaths for holding said penetrating members in a straight configuration prior to deployment.

32. The device of claim 29 wherein said penetrating members assume a curved configuration when extended outward and unconstrained by the ring.

33. The device of claim 29 further comprising a rotary pusher having a plurality of rods which extend radially outward from a center to push said penetrating members when the rotary pusher is rotated.

34. The device of claim 29 further comprising an orientation marker.

35. A method comprising:
accessing a femoral blood vessel and inserting a guidewire to guide valve delivery assembly to a target site in the heart;
advancing an anvil portion of the valve delivery assembly along the guidewire in a collapsed configuration where the anvil is advanced through a valve, wherein the anvil is positioned below a valve annulus;
expanding anvil at the target site into an expanded configuration so that the anvil will engage said valve;
advancing a valve fastener portion of the valve delivery assembly along the guidewire in a collapsed configuration to the target site until the valve fastener portion engages an alignment marker indicating that the desired position has been reached, wherein the valve fastener portion includes a valve excisor;
expanding said valve prosthesis and valve fastener portion to engage tissue at the target site, wherein expanding the fastener assembly advances penetrating members on said fastener into valve tissue, said penetrating members being secured in said valve tissue and the valve prosthesis, and act as anchors to hold said valve prosthesis in position;
moving the valve leaflet support and a valve excisor together to remove leaflets of the valve; and
capturing cut valve leaflets in a prosthesis protective cone.

36. The method of claim 35 further comprising positioning an embolic screen downstream from the valve fastener portion to capture debris.

37. The method of claim 35 further comprising positioning an embolic screen downstream from the valve fastener portion and upstream from arteries of the aortic arch.

38. A kit comprising:
a container;
a first apparatus as set forth in claim 18;
a second apparatus as set forth in claim 18; and
instructions for use setting forth the method of claim 7, wherein said first apparatus, second apparatus, and instructions for use are placed in said container.
